(19) United States
(12) Patent Application Publication
(10) Pub. No.: US 2021/0031073 A1
(43) Pub. Date: Feb. 4, 2021

(54) INSTRUCTIONS OF USING SPORT FITNESS EQUIPMENT COMBINED WITH HUMAN PHYSIOLOGICAL INFORMATION AND SPORTS INFORMATION

(71) Applicant: Ya-Chi CHEN, New Taipei City (TW)

(72) Inventor: Ya-Chi CHEN, New Taipei City (TW)

(21) Appl. No.: 16/525,099

(22) Filed: Jul. 29, 2019

(51) Int. Cl. A63B 24/00; A63B 71/00

(52) U.S. Cl. CPC A63B 24/0075; A63B 71/0054; A63B 2024/0068; A63B 2230/505; A63B 2220/62; A63B 2230/062; A63B 2230/425; A63B 2024/0078

(57) ABSTRACT

Instructions for using sport fitness equipment combined with human physiological information and sports information are provided. The instruction of use is performed by using a sports management device. When a user uses the sport fitness machine to exercise, the sports management device can combine the physiological information of the user with the exercise information of the exercise state of the exercise machine, then provide the user or professional sports and fitness coach to view it any time, as a reference for the user to decide the following sport item or whether the intensity of exercise needs to be adjusted.

### CROSS-REFERENCE TO RELATED U.S. APPLICATIONS

Not applicable.

### STATEMENT REGARDING FEDERALLY SPONSORED RESEARCH OR DEVELOPMENT

Not applicable.

### NAMES OF PARTIES TO A JOINT RESEARCH AGREEMENT

Not applicable.

### REFERENCE TO AN APPENDIX SUBMITTED ON COMPACT DISC

Not applicable.

### BACKGROUND OF THE INVENTION

### 1. Field of the Invention

The present invention relates generally to a field of sports and fitness, and more particularly to the instructions of using the sports fitness equipment combined with human physiological information and sports information.

### 2. Description of Related Art Including Information Disclosed Under 37 CFR 1.97 and 37 CFR 1.98.

Appropriate exercise is conducive to promoting physical health and releasing stress. Limited by working hours, weather or field and other environmental conditions, in recent years, the lifestyle of sports in the gymnasium setting has been gradually developed. Under the guidance of professional coaches, users can use the different types of sports equipment in gymnasium for different forms of exercise based on personal needs.

Existing sports equipment can detect and record the use course information, provide users or coaches with access, also it can be seen that there is a personal wearable sport sensor for sensing and recording personal physiological information such as heartbeat in exercising, providing users with reference of whether the goal of sports is achieved.

After checking, the following problems are still found in the practical application experience of the aforementioned use patterns of existing sports equipment: the gym is provided with a variety of sports equipment, each sports equipment is usually several or even dozens, and there are multiple users to use the multiple identical or different sports equipment for exercising in the gym, and individual user also often use multiple different sports equipment during the same exercise period, the existing sports equipment is unable to distinguish the use state of specific users, the personal wearable sport sensor cannot distinguish the sport type, which is not conducive to that the user or the coach determines whether have the need to strengthen or change the forms of exercise.

### BRIEF SUMMARY OF THE INVENTION

The present invention mainly aims to provide the instructions of using sports fitness equipment combined with human physiological information and sports information, and the technical problem to be solved is how to develop the instructions of the new sports fitness equipment combined with human physiological information and sports information for the innovative breakthrough, which has more ideal practicality.

Based on the foregoing objects, the present invention provides instructions of using the sports fitness equipment combined with physiological information and sports information, which are performed by using a sports management device.

The sports management device includes a user module, an exerciser module and a management module, wherein the user module is disposed in a wearable device of the user for detecting physiological information of the user, and the user module is provided with a user identifier. The exerciser module is disposed in sports fitness equipment for collecting sports information of the sports fitness equipment, and the exerciser module is provided with an exerciser identifier, and the user module and the exerciser module transmit the information in both directions, the exerciser module and the management module transmit the information in both directions.

The instructions include the following steps:

The user is matched with the sports fitness equipment: the user module externally transmits a signal including the user identifier, the exercise module receives the signal, and the exercise module displays the user identifier based on the signal, the user can select to use the sports fitness equipment.

Using the sports fitness equipment: the sports management device determines whether the user uses the sports fitness equipment based on whether the user operates the exerciser module; if the user uses the sports fitness equipment, the exerciser module is operated; if the user does not use the sports fitness equipment, the exerciser module is not operated, and the user may choose to use other sports fitness equipment.

Obtaining information: based on that the user uses the sports fitness equipment, the exerciser module transmits an instruction to the user module, the user module starts to detect the physiological information of the user, and the exerciser module starts to collect the sports information generated by the sports fitness equipment.

Binding information: the user module transmits the physiological information and the user identifier to the exerciser module, and the exerciser module transmits the physiological information, the user identifier, the sports information and the exerciser identifier to the management module.

Storing information: the management module stores the physiological information, the sports information and the exerciser identifier in a database marked with the user identifier, which are used for the user or the sports fitness coach to view.

Stop using the sports fitness equipment: the sports management device determines whether to stop using the sports fitness equipment based on the foregoing binding information step; if stopping to use the sports fitness equipment, the step of obtaining the information is stopped, the user may re-seek the sports fitness equipment matched with the obtained physiological information and the sports information, continuing to perform the binding information step and the storing information step; if the sports fitness equipment is not stopped, the step of obtaining the information is continued.

When the user uses the sports fitness equipment for exercising, the present invention can combine the physiological information of a specific user and the sports information of the specific sports fitness equipment, to provide them to the user or professional sports fitness coach to view at any time, as the reference basis whether the user needs to adjust the choice of sports or the intensity of exercise.

### BRIEF DESCRIPTION OF THE SEVERAL VIEWS OF THE DRAWINGS

FIG. 1 is an online schematic diagram of a sports management device used in the preferred embodiment of the present invention.

FIG. 2 is a flow diagram of a preferred embodiment of the present invention.

FIG. 3 is a circuit block diagram of a user module of a sports management device used in the preferred embodiment of the present invention.

FIG. 4 is a circuit block diagram of an exerciser module of a sports management device used in the preferred embodiment of the present invention.

FIG. 5 is a circuit block diagram of a management module of a sports management device used in a preferred embodiment of the present invention.

### DETAILED DESCRIPTION OF THE INVENTION

The figures illustrate the instructions of using the sports fitness equipment combined with human physiological information and sports information of the present invention, and a sports management device for performing the instructions, but the embodiments are for illustrative purposes only, which is not subject to this structure in the patent application.

The instructions of using the sports fitness equipment combined with human physiological information and sports information of the present invention is performed by using a sports management device.

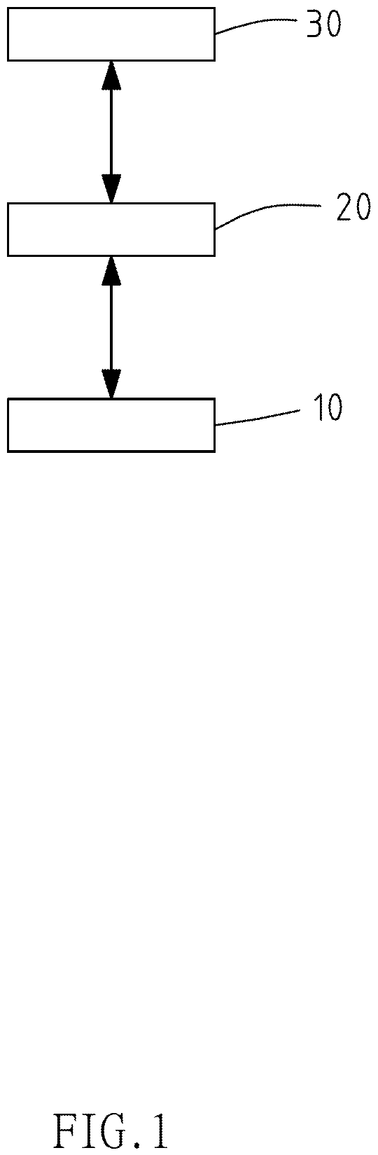

As shown in FIG. 1, the sports management device includes a user module 10, an exerciser module 20 and a management module 30, wherein the user module 10 is disposed in a wearable device (not shown in the figure), and the wearable device can be worn in the user's chest strap, or in the user's wrist support, or in a bind ring on the upper arm of the user or other device that can be worn on the body of the user without hindering the user's sports. The user module 10 is used to detect the physiological information of the user, and the user module 10 is provided with a user identifier. The exerciser module 20 is disposed in a sports fitness equipment (not shown in the figure), and the exerciser module 20 is used to collect the sports information of the sports fitness equipment, and the exerciser module 20 is provided with an exerciser identifier. The user module 10 and the exerciser module 20 transmit the information in both directions, and the exerciser module 20 and the management module 30 transmit the information in both directions.

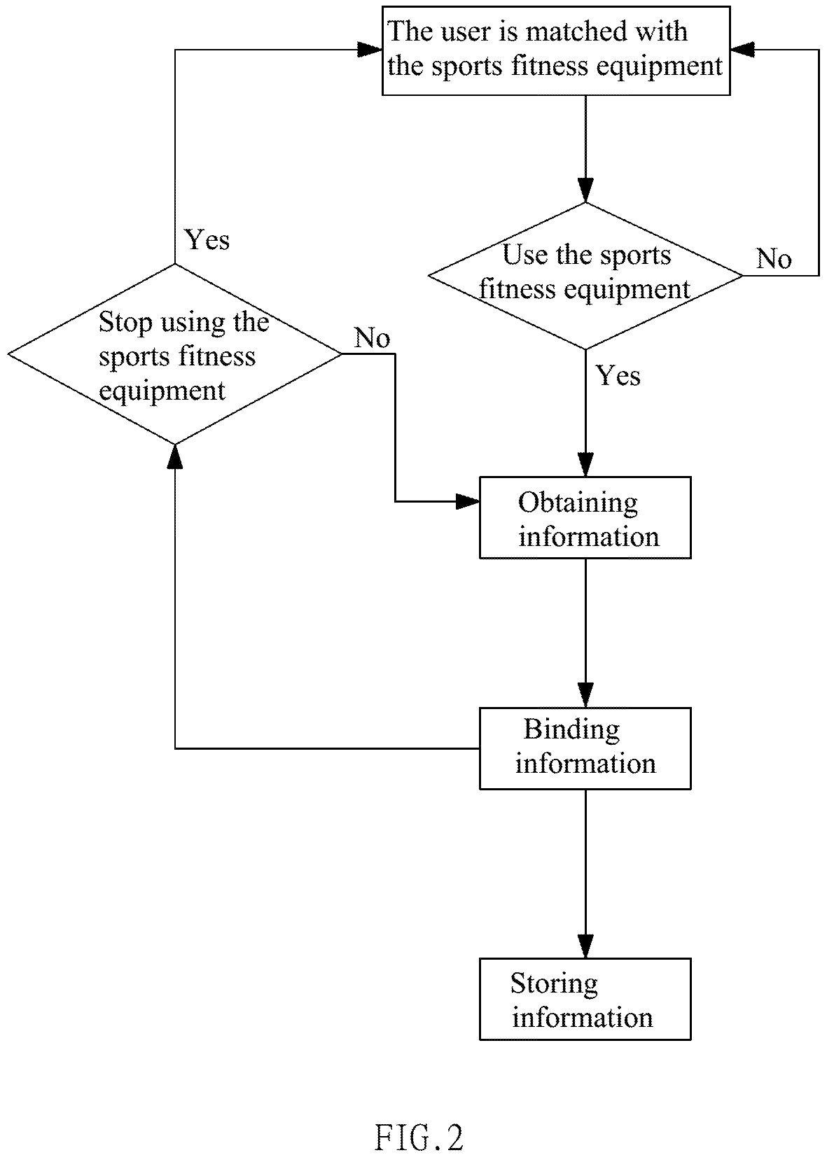

As shown in FIG. 2, the instructions include the following steps:

The user is matched with the sports fitness equipment: the user module 10 externally transmits a signal including the user identifier, the exerciser module 20 receives the signal, and the exerciser module 20 displays the user identifier based on the signal, the user can select to use the sports fitness equipment.

Using the sports fitness equipment: the sports management device determines whether the user uses the sports fitness equipment based on whether the user operates the exerciser module 20; if the user uses the sports fitness equipment, the user operates the exerciser module 20; If the user does not use the sports fitness equipment, the user does not operate the exerciser module 20. At this time, the user may choose to use other sports fitness equipment.

Obtaining information: based on the user operating the exerciser module 20, the sports management device determines whether the user uses the sports fitness equipment, the exerciser module 20 transmits an instruction to the user module 10, and the user module 10 starts to detect the physiological information of the user, and the exerciser module 20 starts to collect the sports information generated by the sports fitness equipment; the physiological information includes one or several values of a heartbeat frequency value, a respiratory frequency value and a body temperature, and the physiological information is not only limited to the heartbeat frequency value, a respiratory frequency value and a body temperature listed above.

Binding information: the user module 10 transmits the physiological information and the user identifier to the exerciser module 20, and the exerciser module 20 transmits the physiological information, the user identifier, the sports information and the exerciser identifier to the management module 30 in an information packet.

Storing information: the management module 30 stores the physiological information, the sports information and the exerciser identifier in a database marked with the user identifier, which are used for the user or the sports fitness coach to view.

Stop using the sports fitness equipment: the sports management device determines whether to stop using the sports fitness equipment based on the foregoing binding information step; if the user stops to use the sports fitness equipment, the step of obtaining the information is stopped, and the foregoing matching step of the user and the sports fitness equipment is performed, so that the user can select to use other sports fitness equipment, and the obtained physiological information and the sports information are continuously stored in the management module 30 by the binding information step and the storing information step before stopping the step of obtaining the information; if the user does not stop using the sports fitness equipment, the step of obtaining the information is continued.

According to the mentioned above, the user uses the sports fitness equipment in exercising, the present combine the physiological information of a specific user and the sports information of the specific sports fitness equipment, to provide them to the user or professional sports fitness coach to view at any time, as the reference basis whether the user needs to adjust the choice of sports or the intensity of exercise.

It is worth mentioning that different user module 10 is respectively provided with different user identifier, and different exerciser module 20 is respectively provided with different exerciser identifier. In the environment of the multiple identical or different form of sports fitness equipment, there are many people engaged in sports at the same time, whether the same sports fitness equipment is used by different users, or the same users use different sports fitness equipment, the generated physiological information and the sports information can be directly bound without confusion, and explicitly stored in the management module 30, which can be easily personalized in management and stored in personalized information of each group of the management module 30, which can show the user's past sports activities, intensity and time, which is beneficial to the user or sports fitness coach to determine whether there is need to strengthen, reduce or change the sports type, so as to meet the user's needs for specific sports purposes, and avoid injury due to excessive exercise, improve the convenience of the user or sports fitness coach for preparing and executing the exercise plan.

Furthermore, before the user is engaged in sports, the personal basic information may be first recorded in the management module 30, and the basic information may include the gender, age, height, weight, body fat percentage, waistline or disease history and other relevant information involving physical function or health status. The sports fitness coach or user can reduce the weight, strengthen heart and lung function, strengthen muscle endurance or shape the body type based on the user's basic information and the user's expected goal. The physiological recommendation value and exercise recommendation value are set in the management module 30. The physiological recommendation value and exercise recommendation value mentioned above can also be set by the management module 30. When users use the sports fitness equipment, the management module 30 will compare the physiological information and exerciser information with the default physiological recommendation value and the preset exercise recommendation value, a recommendation information is transmitted to the exerciser module 20 based on the results of the comparison, providing the user with a reference for subsequent exercise.

Furthermore, in the state that the binding information step is executed, the management module 30 compares the plural sports information continuously generated in the default time interval with the default stop information, if each sports information in the time interval respectively accords with the stop information, and the management module 30 determines that the user stops using the sports fitness equipment, and the time interval is 3-60 seconds, and the stop information is determined by the information that the sports fitness equipment is not used.

For example, when the user uses the bicycle exerciser to exercise, the sports information of the bicycle fitness equipment is the speed of the flywheel (Unit: RPM), and the stop information is 0 RPM, and the sports information is in a time interval of 3 seconds. When both are 0 RPM, the management module 30 determines that the user stops using the bicycle fitness equipment.

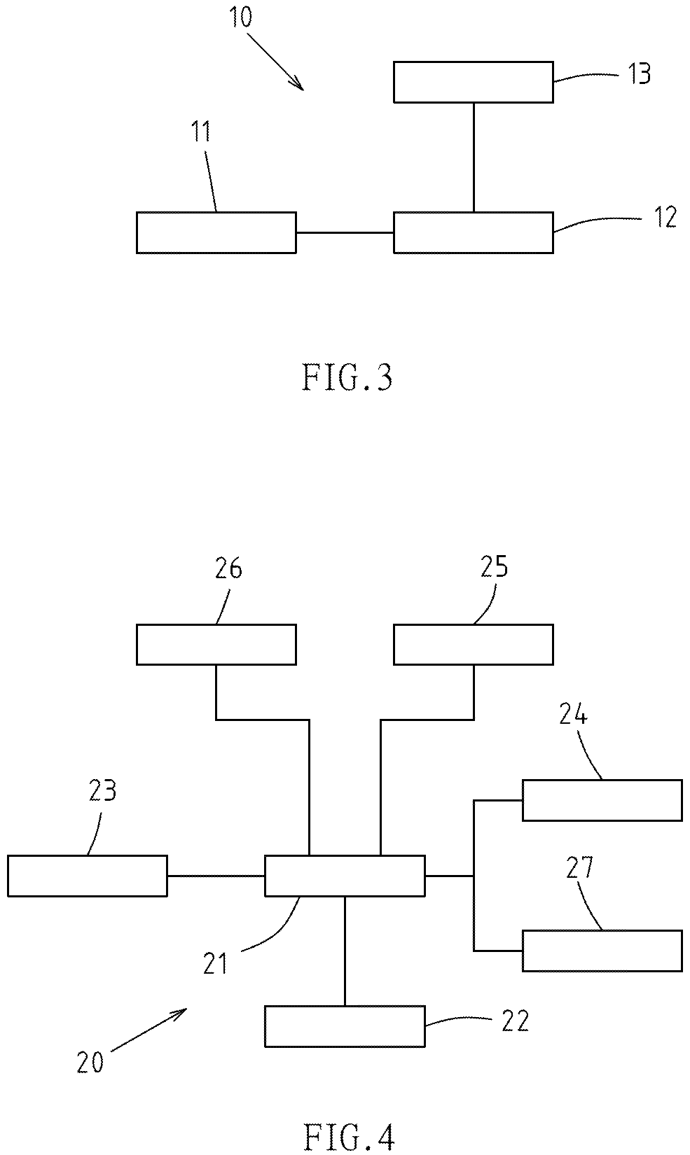

As shown in FIG. 1 and FIG. 3, the user module 10 includes a physiological information collector 11, a first microcontroller 12 and a first wireless communicator 13, wherein the physiological information collector 11 and the first wireless communicator 13 are respectively coupled with the first microcontroller 12, the first microcontroller 12 respectively controls the physiological information collector 11 and the first wireless communicator 13, The physiological information collector 11 is used to sense the physiological information of the user and transmits the physiological information to the first microcontroller 12, the first microcontroller 12 is a microcontroller (Micro Controller Unit, MCU), the first microcontroller 12 is internally provided with the user identifier, the first microcontroller 12 transmits the user identifier and the physiological information to the first wireless communicator 13, the first wireless communicator 13 is used to transmit outwards the user identifier and the physiological information and receive the instruction given by the exerciser module 20.

As shown in FIG. 1 and FIG. 4, the exerciser module 20 includes a second microcontroller 21, a second wireless communicator 22, a sports information collector 23, a display 24, a startup unit 25 and a stop unit 26, wherein the second wireless communicator 22, the sports information collector 23, the display 24, the startup unit 25 and the stop unit 26 are respectively coupled with the second microcontroller 21, so that the second microphone 21 respectively controls the second wireless communicator 22, the sports information collector 23 and the display 24. The sports information collector 23 is used to collect the sports information of the sports fitness equipment, and transmit the sports information to the second microcontroller 21. The second microcontroller 21 is a microcontroller, and the second microcontroller 21 is internally provided with exerciser identifier. The second wireless communicator 22 is used to receive the user identifier, the physiological information and the management information transmitted by the management module 30, and the user identifier, the physiological information, the sports information and the exerciser identifier are transmitted to the management module 30, an instruction is transmitted to the user module 10, and the display 24 is used to display the status of the sports fitness equipment, the startup unit 25 is a control loop for operation of the user. Hereby, a control instruction is transmitted to the second microcontroller 21, and the exerciser module 20 transmits an instruction to the user module 10, and receives the physiological information transmitted by the user module 10. The stop unit 26 is a control loop for operation of the user, hereby a control instruction is transmitted to the second microcontroller 21 for controlling the exerciser module 20 to transmit an instruction to the user module 10 and interrupting the physiological information transmitted by the user module 10.

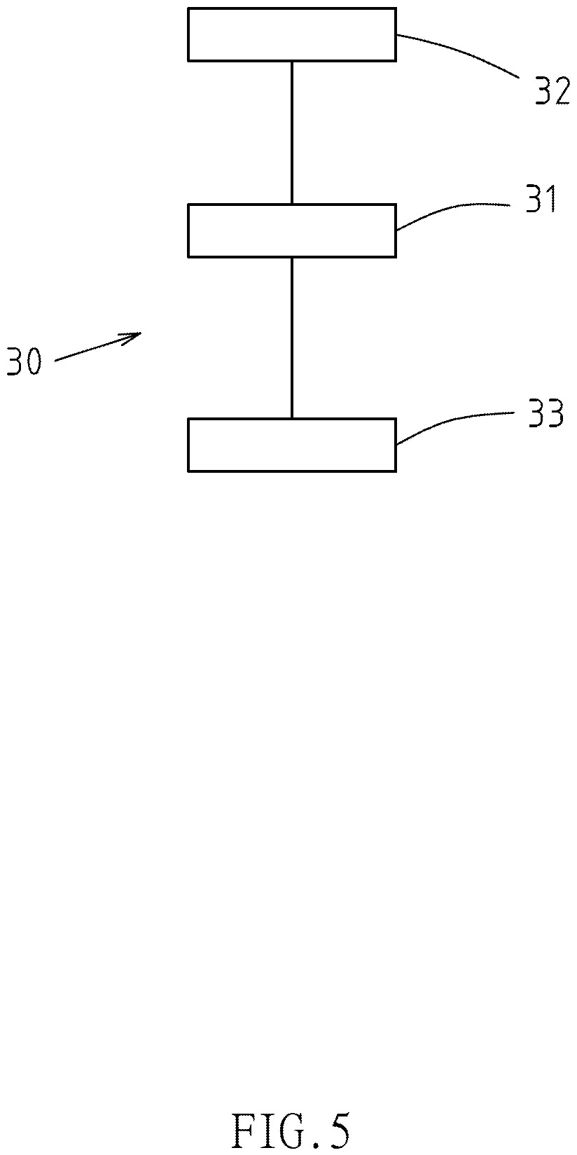

As shown in FIG. 1 and FIG. 5, the management module 30 includes a third microcontroller 31, a third wireless communicator 32 and a storage 33, wherein the third microcontroller 31 is coupled with the third wireless communicator 32 and the storage 33, so that the third microcontroller 31 can control the third wireless communicator 32. The third wireless communicator 32 is used to receive the user identifier, the physiological information, the sports information and the executive identifier, and transmit the management information, the third microcontroller 31 is a microcontroller for processing the user identifier, the physiological information, the sports information and the exerciser identifier, and storing the user identifier, the physiological information, the sports information and the exerciser identifier stored and processed in the storage unit 33.

In the matching step of the user and the sports fitness equipment, the exerciser module 20 can display the user identifier on the display 24, and provide the use step of the sports fitness equipment for the user, select whether to operate the startup unit 25, start using the sports fitness equipment, and the user can operate the startup unit 25 by selecting an icon displayed on the display 24.

During the use of the sports fitness equipment, the display 24 can be used to display the physiological information and the sports information, and further display the recommendation information transmitted by the management module 30, providing the user with real-time sports state, as the reference whether needing to adjust the sport item or exercise intensity.

The step to stop using the sports fitness equipment is used to determine whether the user may choose to actively operate the stop unit 26 when stopping to use the sports fitness equipment, so that the sports management device determines to stop using the sports fitness equipment.

In the step to stop using the sports fitness equipment, the exerciser module 20 can display the icon of the stop use on the display 24, provide the user to select the click, operate the stop unit 26, and stop using the sports fitness equipment.

The management module 30 stores the processed user identifier, the physiological information, the sports information and the exercise identifier in the storage 33. According to the user identifier and through the third wireless communicator 32, the physiological information and the sports information are actively transmitted to an external device or cloud that is authorized in advance, and the external device may be a mobile communication device used by the user or a mobile communication device used by the sports fitness coach or a management system of the sports fitness center, the information is provided to the user or the sports fitness coach to determine whether the sport item or intensity is further adjusted.

As shown in FIG. 4, the exerciser module 20 is further provided with a warning indicator 27, and the warning indicator 27 is coupled to the second microcontroller 21. The instructions of the present invention may further include a warning step (not shown in the figure), wherein the management module 30 compares the physiological information with a default warning threshold, if the value of the physiological information is higher than or lower than the warning threshold, the management module 30 transmits a signal to the exerciser module 20, the second microcontroller 21 controls the warning indicator 27 to generate a warning information, so as to prompt the user to suspend the exercise or to carry out the gradual and slow exercise. Someone else in the environment of the sports fitness equipment can also promptly take corresponding assistance or rescue measures to reduce the accident risk through the warning information, improve the sports fitness safety; the warning information can be displayed on the display 24 of the exerciser module 20 as needed.

I claim:

1. The instructions of using sports fitness equipment combined with human physiological information and sports information, the instructions is performed by a sports management device;
the sports management device includes a user module, an exercise module and a management module, wherein the user module is disposed in a wearable device of the user for detecting physiological information of the user, and the user module is provided with a user identifier;
the exerciser module is disposed in sports fitness equipment for collecting sports information of the sports fitness equipment, and the exerciser module is provided with an exerciser identifier, and the user module and the exerciser module transmit the information in both directions, the exerciser module and the management module transmit the information in both directions;
the instructions include the following steps:
the user is matched with the sports fitness equipment: the user module externally transmits a signal including the user identifier, the exercise module receives the signal, and the exercise module displays the user identifier based on the signal, the user can select to use the sports fitness equipment;
using the sports fitness equipment: the sports management device determines whether the user uses the sports fitness equipment based on whether the user operates the exerciser module; if the user uses the sports fitness equipment, the exerciser module is operated; if the user does not use the sports fitness equipment, the exerciser module is not operated, and the user may choose to use other sports fitness equipment;
obtaining information: based on that the user uses the sports fitness equipment, the exerciser module transmits an instruction to the user module, the user module starts to detect the physiological information of the user, and the exerciser module starts to collect the sports information generated by the sports fitness equipment;
binding information: the user module transmits the physiological information and the user identifier to the exerciser module, and the exerciser module transmits the physiological information, the user identifier, the sports information and the exerciser identifier to the management module;
storing information: the management module stores the physiological information, the sports information and the exerciser identifier in a database marked with the user identifier, which are used for the user or the sports fitness coach to view;
stop using the sports fitness equipment: the sports management device determines whether to stop using the sports fitness equipment based on the foregoing binding information step; if stopping to use the sports fitness equipment, the step of obtaining the information is stopped, the user may re-seek the sports fitness equipment matched with the obtained physiological information and the sports information, continuing to perform the binding information step and the storing information step; if the sports fitness equipment is not stopped, the step of obtaining the information is continued.

2. The instructions of using sports fitness equipment combined with human physiological information and sports information defined in claim 1, wherein the exerciser module includes a startup unit, based on that the user operates the startup unit as the basis of the sports management device to determine using the sports fitness equipment.

3. The instructions of using sports fitness equipment combined with human physiological information and sports information defined in claim 1, wherein the exerciser module includes a stop unit, and based on that the user operates the stop unit, the sports management device determines to stop using the sports fitness equipment.

4. The instructions of using sports fitness equipment combined with human physiological information and sports information defined in claim 2, wherein the exerciser module includes a stop unit, and based on that the user operates the stop unit, the sports management device determines to stop using the sports fitness equipment.

5. The instructions of using sports fitness equipment combined with human physiological information and sports information defined in claim 3, wherein the management module compares the plural sports information continuously generated in the default time interval with the default stop information, if each sports information in the time interval respectively accords with the stop information, and the management module determines that the user stops using the sports fitness equipment.

6. The instructions of using sports fitness equipment combined with human physiological information and sports information defined in claim 4, wherein the management module compares the plural sports information continuously generated in the default time interval with the default stop information, if each sports information in the time interval respectively accords with the stop information, and the management module determines that the user stops using the sports fitness equipment.

7. The instructions of using sports fitness equipment combined with human physiological information and sports information defined in claim 5, wherein the time interval is 3-60 seconds.

8. The instructions of using sports fitness equipment combined with human physiological information and sports information defined in claim 6, wherein the time interval is 3-60 seconds.

9. The instructions of using sports fitness equipment combined with human physiological information and sports information defined in claim 1, wherein the exerciser module is provided with a warning indicator, the instructions further includes a warning step, wherein the management module compares the physiological information with a default warning threshold, if the value of the physiological information is higher than or lower than the warning threshold, the warning indicator generates a warning information, thereby improving the sports fitness safety.

10. The instructions of using sports fitness equipment combined with human physiological information and sports information defined in claim 1, wherein the management module will compare the physiological information and exerciser information with the default physiological recommendation value and the preset exercise recommendation value, the recommendation information is transmitted to the exerciser module based on the results of the comparison, providing the user with a reference for subsequent exercise.

11. The instructions of using sports fitness equipment combined with human physiological information and sports information defined in claim 10, wherein the physiological recommendation value and the sports recommendation value are set according to the user's sports goal.

12. A sports management device, wherein the sports management device is used to implement the instructions of the sports fitness equipment combined with human physiological information and sports information as described in claim 1.
